(19) United States
(12) Patent Application Publication
(10) Pub. No.: US 2005/0043539 A1
(43) Pub. Date: Feb. 24, 2005

(54) Process for the preparation of 5-[4-[2-[N-methyl-N-(2-pyridyl) amino] ethoxy] phenyl methyl] thiazolidine-2, 4-dione maleate

(76) Inventors: Lalji Karsan Gediya, Mumbai (IN); Venkatasubramanian Radha Tarur, Mumbai (IN); Suresh Mahadev Kadam, Navi Mumbai (IN); Subodh Shashikant Patnekar, Thane (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/938,317

(22) Filed: Sep. 10, 2004

Related U.S. Application Data
(63) Continuation-in-part of application No. 10/431,847, filed on May 7, 2003.

(30) Foreign Application Priority Data
Jan. 28, 2004 (IN) 80/MUM/2004

(51) Int. Cl. C07D 417/02

(52) U.S. Cl. 546/269.7

(57) ABSTRACT

The present invention discloses a process for the preparation of 5-[4-[2-[N-methyl-N-(2-pyridyl)amino]ethoxy]phenyl methyl]thiazolidine-2,4-dione maleate (VI) comprising the steps of Coupling 2-[N-methyl-N-(2-pyridyl)amino]ethanol (I) and 4-fluorobenzaldehyde (II) in N,N-dimethylformamide, isolating the coupled product 4[2-[N-methyl-N-(2-pyridyl)amino]ethoxy]benzaldehyde (III), converting said isolated benzaldehyde compound (III) to 5-[4-[2-[N-methyl-N-(2-pyridyl)amino]ethoxy]benzylidene]thiazolidine-2,4-dione (IV) and purifying the same, reducing 5-[4-[2-[N-methyl-N-(2-pyridyl)amino]ethoxy]benzylidene]thiazolidine-2,4-dione, by a novel reduction method for making 5-[4-[2-[N-methyl-N-(2-pyridyl)amino]ethoxy]phenyl methyl]thiazolidine-2,4-dione (V). This reduction method involves reacting the compound (IV) with a novel metal legand complex and a reducing agent, purifying the product (V) obtained by a new method reported in the present invention and converting the said thiazolidine-2,4-dione compound (V) into a pharmaceutically acceptable salt.

### FIELD OF THE INVENTION

The present invention relates to a novel process for the preparation of 5-[4-[2-[N-methyl-N-(2-pyridyl)amino]ethoxy]phenyl methyl]thiazolidine-2,4-dione] (V) known as rosiglitazone, an antidiabetic compound , which is the drug of choice for non-insulin dependant diabetes mellitus (NIDDM). The invention further relates to the novel process of reduction and subsequent purification, which results into substantially pure rosiglitazone and its salts in better yields.

### BACKGROUND AND PRIOR ART

U.S. Pat. No. 5,002,953 discloses the process for reducing the 5-[4-[2-[N-methyl-N-(2-pyridyl)amino]ethoxy]benzylidene]thiazolidine-2,4-dione (IV) to 5-[4-[2-[N-methyl-N-2-(pyridyl)amino]ethoxy]phenyl methyl]thiazolidine-2,4-dione (V) by using hydrogen on palladium catalyst in 1,4-dioxane. Such process that involves use of noble metal is always costly as it involves use of noble metal. Secondly it has inherent problems of safety as noble metal is used. Yield and poisoning of catalyst are other issues, which make it a secondary choice. WO 9923095 relates to similar process in glacial acetic acid.

Bioorganic Medicinal Chemistry Letters, 1994, Vol. 4, 1181-84 discloses the use of Magnesium metal & Methanol for reduction of 5-[4-[2-[N-methyl-N-(2-pyridyl)amino]ethoxy]benzylidine]thiazolidine-2,4-dione (IV) to 5-[4-[2-[N-Methyl-N-(2-Pyridyl)amino]ethoxy]phenyl methyl]thiazolidine-2,4-dione(V). Use of large quantities of Magnesium metal, formation of alkoxide with methanol is inherent drawbacks of this process, which necessitate a better option if available. Other associated drawbacks include uncontrolled evolution of hydrogen and therefore safety issues, removal of Magnesium alcoholate from Methanol, discoloration. WO 93, 93/10254 relates to bio-transformation by Rhodoturola Yeast for conversion of 5-[4-[2-[N-methyl-N-(2-pyridyl)amino]ethoxy]benzylidine]thiazolidine-2,4-dione (IV) to 5-[4-[2-[N-methyl-N-2-(pyridyl)amino]ethoxy]phenyl methyl]thiazolidine-2,4-dione (V). Such biotransformations always involve lot of capital expenditure and process is highly sensitive and therefore prone for failures. Precise controls and sensitivity being main drawbacks.

WO 98/37073 provides a reduction method using Lithium borohydride/THF/Pyridine/NaBH₄/LiCl/Pyridine and Lithium tri-s-butyl borohydride.

U.S. Pat. No. 5,002,953 and WO 99/23095 disclose reduction of double bond for the preparation of 5-[4-[2-[N-methyl-N-(2-pyridyl)amino]ethoxy]phenyl methyl]thiazolidine-2,4-dione (V). The disclosure has inherent drawbacks. It involves a troublesome step, requires high-pressure hydrogenation using palladium supported on carbon catalyst. In this process high amount of palladium was required which indirectly enhances the cost as well as safety concerns i.e. while handling the catalyst. Also the yield was about 70-80%. In the said process poisoning of catalyst was observed due to thiazolidinedione moiety containing sulphur and hence at times reaction needed longer time for completion.

In case of metal reduction (Reported in Bio. Med. Chem. Lett. 1994, Vol 4, 1181-84) large quantity of magnesium metal is required, as it forms alkoxide with methanol aggravating the work up procedure making it more tedious and cumbersome. Further usage of excess magnesium in methanol causes uncontrolled evolution of hydrogen that can lead to safety hazards. Lastly the removal of magnesium alcoholate of methanol from the 5-[4-[2-[N-methyl-N-(2-pyridyl)amino]ethoxy]phenyl methyl]thiazolidine-2,4-dione (V) is difficult, yielding lower efficiency and in turn imparts colour to the final product.

WO 98/37073 Disclosing biotransformation of a 5-[4-[2-[N-methyl-N-(2-pyridyl)amino ethoxy]benzylidene]thiazolidine-2,4-dione (IV) to its corresponding benzyl derivative was reported by Rhodotorula rubra. However it is time consuming and difficult to implement on the plant scale, requiring highly sophisticated infrastructure to grow the enzyme.

The following references disclose cobalt catalyzed reductions:

Leutenegger U. Leutengga et. al. Angew. Chem. Int. Ed., 28: 60 (1989) discloses enantioselective reduction of α,β-unsaturated carboxylates with sodium borohydride and catalytic amounts of chiral cobalt semi -corrin complexes.

M. N. Ricroch and A. Gandemer J. Organometal. Chem. 67 : 119 (1974) discloses (pyridinalo) cobaloxime , chloro(pyridinalo)cobaloxime and Vitamin B₁₂ catalysing, the hydrogenation of α,β-unsaturated esters by hydrogen or sodium borohydride.

J. O. Oshy et al (J Amer Chem Soc) 108:67-72 (1986) discloses cobalt(II) mediated sodium borohydride and lithium aluminium hydride reductions, which do not involve the use of ligands.

In the penultimate steps the formation of the pharmaceutically acceptable maleate salt is reported by Cantello et al J med Chem., 1994, 37 3977-3985 in methanol. Yield 62%. Pool et al (WO 94/05659) have prepared the maleate in ethanol.

WO 064892, 1999 relates preparation of maleate using ethanol/water mixture. Use of denatured ethanol (5% v/v methanol) is mentioned in the patent, WO 064893. WO 064896 describes preparation of maleate in acetone under N₂.

In all the above reported inventions, pure maleate salt is obtained using mixture of solvents in 75-90% yield.

Various solvents are being industrially used for various processes that are process specific, product specific. Many a times an attempt to use different class of solvents frustrates the purpose. Such classifications are often being given on the basis of polarity, behavioral characteristics, number of polar positions present in the molecule and so on.

Another way to represent the same includes use of dielectric constants values of the solvents. The representative chart of solvents with their dielectric constants is listed in Handbook of Chemistry and Physics, by David R. Lide 81^{st} Edition Page 6-149 to 6-171 for reference.

The prior art reported above for the reduction of the benzylidene compound (IV) can be said to have following not so favorable attributes which if overcome would be of immense industrial advantage.
1) U.S. Pat. No. 5,002,953 and WO 99/23095 uses palladium, which is very expensive, not so safe and hazardous.
2) Mg metal in methanol (Reported in Bio. Med. Chem. Lett. 1994, Vol 4, 1181-84) has the inherent problem of difficulty to control the reaction during scale up.
3) Bio transformation requires special infrastructure and
4) WO 98/37073 uses LiBH4, which is extremely expensive

### OBJECTS OF INVENTION

1. The main object of the present invention is to provide a novel and an industrially viable and cost effective process for the preparation of rosiglitazone maleate which obviates the drawbacks of prior art process by use of cheaper & easily available raw-materials.
2. Another aspect of the invention is to provide 5-[4-[2-[N-methyl-N-(2-pyridyl)amino]ethoxy]phenyl methyl]thiazolidene-2,4-dione (V) in high yield & purity by reduction of 5-[4-[2-[N-methyl-N-2-(pyridyl)amino]ethoxy]benzylidene]thiazolidine-2,4-dione (IV).
3. Yet another aspect of this invention is to provide purification method for intermediate (IV) and (V) in order to achieve high purity.
4. Yet another objective of the present invention is to obtain the pharmaceutically acceptable salt, viz. rosiglitazone maleate from rosiglitazone base in high yield and purity using Acetone\IPA mixture.

### SUMMARY OF THE INVENTION

The present invention provides a process for reducing 5-[4-[2-[N-methyl-N-(2-pyridyl)amino]ethoxy]benzylidine]thiazolidine-2,4 dione (IV) to 5-[4-[2-[N-methyl-N-(2-pyridyl)amino]ethoxy]phenyl methyl]thiazolidine 2,4-dione (V) using cobalt ion, a ligand and a reducing agent. This process employs temperature in the range of 20-45° C. and wherein a suitable solvent which is mixture of solvents is used, viz. THF/DMF/Water.

The novel purification route selected gives substantially pure yield. Conventionally inorganic metal when loosely bonded to organic substrate, the adduct is called complex. In the present invention ammonia forms a loose adduct with organic substrate, which is referred as complex and be construed accordingly.

### DETAILED DESCRIPTION OF THE INVENTION

The present invention offers a novel reduction method, which is more efficient because it is faster, easier and results in substantially improved yield of the desired product. It is also more convenient for scale up at plant, since no high-pressure autoclaves are required. The solvents used for this process are THF, DMF and water by alone or a mixture thereof.

Various solvents with different constitution are used which are process specific, product specific. Hydroxylic solvents are to be construed to mean solvents whose molecular formula has hydroxyl group as electronegative part of molecule. Solvents used in purification step in the present invention have been exemplified in examples number 1 & 2. They belong to class of lower carbon chain. The lower carbon chain alcohols are to be construed to mean those alcohols whose molecular formula has number of carbons in the range of C₁ to C₄. These can be present in the straight chain without branching or carbon atoms may align in such a way that they are not present in straight chain but the molecule has branched arrangement of carbons atoms. Example of such solvent can be isopropyl alcohol.

The process of reduction facilitates use of diverse solvents including solvents with heteroatoms present in the molecular formula.

Use of Ligand and complexing agent employed for the above process is adequately elaborated in examples e.g. example no. 2.

Use of borohydride of alkali metal gives an impressive yield. The process is illustrated in example no. 2. In the said example the reducing agent used is sodium borohydride. The yield is high in the range of 90-95%, yielding product with a purity of about 97% by HPLC.

Alcoholic purification of intermediate (IV) enhances purity of (V) to 97 to 97.5% by HPLC where as the crude product has purity in the range of 88-90%. The process of purification of IV is illustrated in example no. 1.

Thus purification of 5-[4-[2-[N-methyl-N-(2-pyridyl)amino]ethoxy]phenyl methyl]thiazolidine-2,4-dione (V) was accomplished by dissolving it in alcohol at alkaline pH obtained by purging dry ammonia gas. This on subsequent neutralization with acetic acid yielded pure compound purity 99% by HPLC.

Further preparation of maleate with purified base (V) provides the final product i.e. 5-[4-[2-[N-methyl-N-(2-pyridyl)amino]ethoxy]phenyl methyl]thiazolidine-2,4-dione maleate (VI) with impurity level below 0.1% as per ICH standards & other international regulations.

The present invention discloses a process for the preparation of 5-[4-[2-[N-methyl-N-(2-pyridyl)amino]ethoxy]phenyl methyl]thiazolidine-2,4-dione maleate (VI) comprising the steps of:
1) Coupling 2-[N-methyl-N-(2-pyridyl)amino]ethanol (I) and 4-fluorobenzaldehyde (II) in N,N-dimethylformamide with sodium hydride as a base.
2) Isolating the coupled product 4-[2-[N-methyl-N-(2-pyridyl)amino]ethoxy]benzaldehyde (III).
3) Converting said isolated benzaldehyde compound (III) to 5-[4-[2-[N-methyl-N-(2-pyridyl)amino]ethoxy]benzylidene]thiazolidine-2,4-dione (IV) in known manner.
4) Purifying the coupled product [IV] with methanol.
5) Reducing 5-[4-[2-[N-methyl-N-(2-pyridyl)amino]ethoxy]benzylidene]thiazolidine-2,4-dione (IV) by a novel reduction method for making 5-[4-[2-[N-methyl-N-(2-pyridyl)amino]ethoxy]phenylmethyl]thiazolidine-2,4-dione (V). This reduction method involves reacting the compound (IV) with a cobalt ion, a ligand and a reducing agent.
6) Purifying the product (V) obtained by a new method reported in the present invention, which comprises of treatment with alcoholic ammonia.
7) Converting the said thiazolidine-2,4-dione compound (V) into a pharmaceutically acceptable salt in mixture of solvents viz Isopropyl alcohol. and Acetone.

### Experimental Findings

### EXAMPLE 1

Purification of compound 5-[4-[2-[N-methyl-N-(2-pyridyl)amino]ethoxy]benzylidene]thiazolidine-2,4-dione (IV):

To a 100 ml 3necked round bottom flask, equipped with a mechanical stirrer is charged 10 gms of compound of formula 5-[4-[2-[N-methyl-N-(2-pyridyl)amino]ethoxy]benzylidene]thiazolidine-2,4-dione (IV). To this 25 ml of methanol is added. The whole solution is refluxed for 1 hour. The reaction mass is then cooled at 10° C., stirred for 1 hour, filtered, washed with 25 ml of cold methanol and dried at 70° C. for 6hrs. Yield of product (IV) is 8gms. Purity is 97% by HPLC.

### EXAMPLE 2

Preparation of compound 5-[4-[2-[N-methyl-N-(2-pyridyl)amino]ethoxy]phenyl methyl]thiazolidine-2,4-dione (V):

To a 100 ml 3necked round bottom flask, equipped with a mechanical stirrer is charged 10 gms of compound (IV), 140 ml of water, 34 ml of tetrahydrofuran and 12 ml of 1.0 N sodium hydroxide. The mixture is stirred at 25° C. for 10 min and cooled to 15° C. To the cooled mixture is added 30 ml of catalyst solution, prepared by dissolving 1.3 gms of dimethylglyoxime and 0.068 gm of cobaltous chloride hexahydrate in 28 ml of dimethylformamide. Then solution of 28.20 ml of sodium hydroxide soln with 20 ml of water is added at the rate of 0.1 ml/min. The reaction is stirred at 15° C. for 4 hours. The reaction is neutralised with 8-10 ml of acetic acid. Solid precipitated out is quenched in 50 ml of water. Solid product is filtered, washed with 50 ml of water and dried. Yield is 9.2 gms (91.5%). Purity by HPLC is 97.5%.

### EXAMPLE 3

Purification of 5-[4-[2-[N-methyl-N-(2-pyridyl)amino]ethoxy]phenylmethyl]thiazolidine-2,4-dione (V) by alcoholic ammonia:

To a 100 ml 3-necked round bottom flask, equipped with a mechanical stirrer is charged 10 gms of compound 5-[4-[2-[N-methyl-N-(2-pyridyl)amino]ethoxy]phenyl methyl]thiazolidine-2,4-dione (V). The compound is taken in a 50 ml methanol and 20 ml of ethanol, cooled it to 10-15° C. Dry ammonia gas is purged in the reaction mixture till the solution became clear solution which further stirred for 10-15 min. 5% charcoal is added, stirred for half an hour and filtered through hy-flo bed. The reaction mixture is cooled to 10-15° C. Acetic acid is added drop wise within 30-35 min maintaining temperature 10-15° C. The solid product is precipitated at pH 6-6.6 which is filtered and washed with 25 ml of cold methanol (10° C.). The product is centrifuged and dried at 65° C. for 6 hrs. Yield is 9 gms (90%). Purity by HPLC is 99%.

### EXAMPLE 4

Preparation of 5-[4-[2-[N-methyl-N-(2-pyridyl)amino]ethoxy]phenyl methyl]thiazolidine-2,4-dione maleate (VI):

To a 500 ml 3-necked round bottom flask, equipped with a mechanical stirrer is charged 10 gms of 5-[4-[2-[N-methyl-N-(2-pyridyl)amino]ethoxy]phenyl methyl]thiazolidine-2,4-dione (V) and 3.5 gms of Maleic acid . Slowly 80 ml of acetone is added in the mixture. Further add 80 ml of IPA in the solution. Separated solid is filtered, washed with 40-50 ml of IPA and dried. Yield is 10.5 gms (80%). Purity by HPLC is 99.5%.

While the present invention is described above in connection with preferred or illustrative embodiments, these embodiments are not intended to be exhaustive or limiting of the invention. Rather, the invention is intended to cover all alternatives, modifications and equivalents included within its scope, as defined by the appended claims.

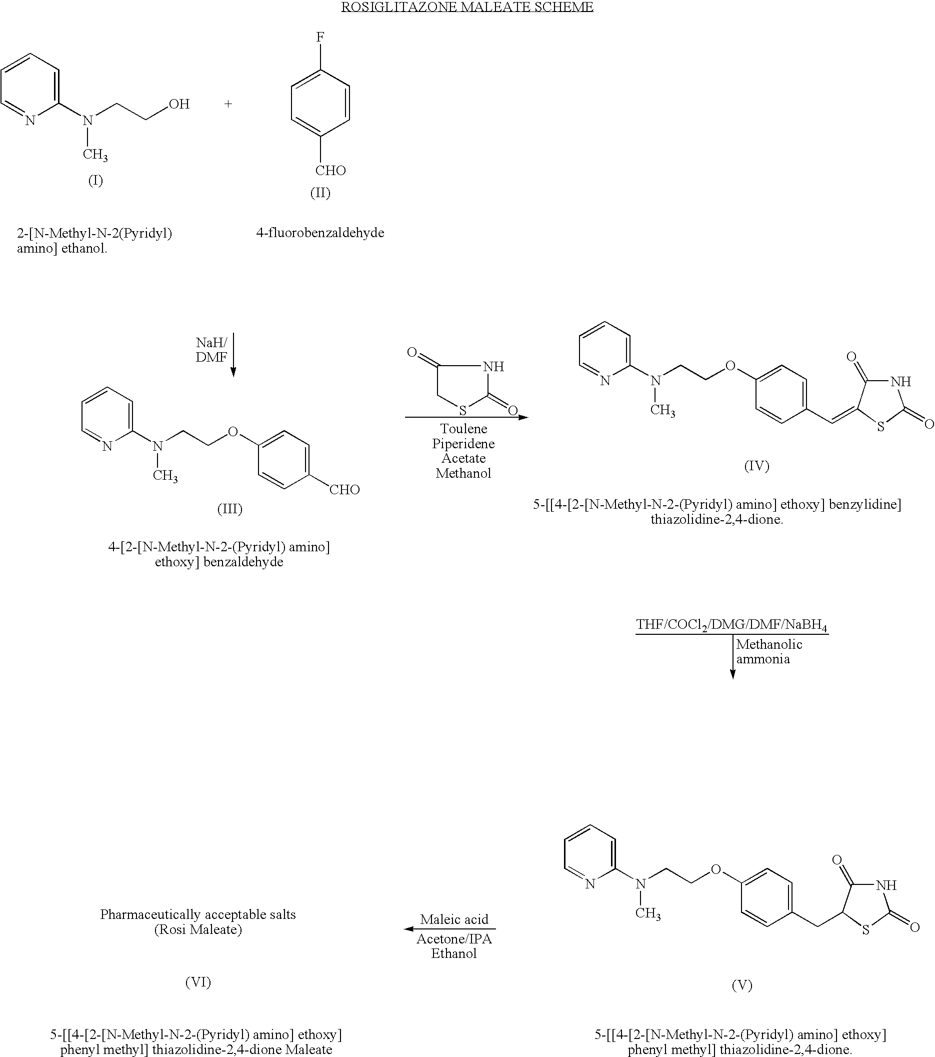

1. Novel process for the preparation of 5-[4-[2-N-methyl-N-(2-pyridyl)amino]ethoxy]phenylmethyl]thiozolidine-2,4-dione maleate (rosiglitazone maleate) wherein the said process comprises;
(a) purifying 5-[4-[2-[N-methyl-N-(2-pyridyl)amino]ethoxy]benzylidene]thiazolidine-2,4-dione (IV) in hydroxylic solvent,
(b) reducing 5-[4-[2-[N-methyl-N-(2-pyridyl)amino]ethoxy]benzylidene]thiazolidine-2,4-dione (IV) with a metal ion, a ligand and a reducing agent,
(c) purifying the product 5-[4-[2-[N-methyl-N-(2-pyridyl)amino]ethoxy]phenyl methyl]thiazolidine-2,4-dione (V) by treating with alcoholic ammonia,
(d) converting the said 5-[4-[2-[N-methyl-N-(2-pyridyl)amino]ethoxy]phenyl methyl]thiazolidine-2,4-dione compound (V) into a pharmaceutically acceptable salt in a mixture of solvents.

2. The process for purification of 5-[4-[2-[N-methyl-N-(2-pyridyl)amino]ethoxy]benzylidene]thiazolidine-2,4-dione (IV) as claimed in claim 1(a) wherein the said hydroxylic solvents are alcohols.

3. The process for purification of 5-[4-[2-[N-methyl-N-(2-pyridyl)amino]ethoxy]benzylidene]thiazolidine-2,4-dione (IV) as claimed in claims 1(a) and 2 wherein the said hydroxylic solvents are preferably alcohols of lower carbon chain.

4. The process for purification of 5-[4-[2-[N-methyl-N-(2-pyridyl)amino]ethoxy]benzylidene]thiazolidine-2,4-dione (IV) as claimed in claims 1(a) and 2 to 3 wherein the most preferred hydroxylic solvents are selected from C1-C4 aliphatic alcohols, including branched chain alcohols.

5. A process for reduction of 5-[4-[2-[N-methyl-N-(2-pyridyl)amino]ethoxy]benzylidene]thiazolidine-2,4-dione (IV) as claimed in claim 1(b) wherein the said reduction is carried out with metal ligand complex and a reducing agent in a solvents at a controlled temperature ranging from 10-15° C. under alkaline conditions of pH in the range of 9 to 11.

6. A process as claimed in claims 1(b) and 5 wherein the said metal ion of the metal ligand complex is selected from bivalent metals.

7. A process as claimed in claims 1(b) and 5 to 6 wherein the preferred bivalent metal of metal ligand complex is cobalt.

8. A process as claimed in claims 1(b) and 5 to 7 wherein the most preferred form of cobalt is cobalt chloride.

9. A process as claimed in claims 1(b) and 5 to 8 wherein the optional form of cobalt is cobalt diacetate.

10. A process as claimed in claim 1(b) wherein the said ligand is an aromatic or aliphatic ligand.

11. A process as claimed in claim 1(b), 5 and 10 wherein the said ligand is bidentate.

12. A process as claimed in claim 1(b), 5 and 10 to 11 wherein the said ligand is preferably dimethyl glyoxime.

13. A process as claimed in claim 1(b), 5 and 10 to 12 wherein the said ligand is optionally 2,2′-bipyridyl.

14. A process as claimed in claim 1(b) and 5 wherein the said potential reducing agent is hydride of group III metal with alkali metal.

15. A process as claimed in claim 1(b), 5 and 14 wherein the most preferred hydride is hydride of boron with alkali metal.

16. A process as claimed in claim 1(b), 5 and 14 to 15 wherein the most preferred borohydride is sodium borohydride.

17. A process as claimed in claim 1(b), 5 and 14 to 16 wherein the most preferred being potassium borohydride and the preferred being lithium borohydride.

18. A process as claimed in claim 1(b), 5 and 14 to 17 wherein the said reducing agent is optionally lithium aluminium hydride.

19. A process as claimed in claim 1(b) and 5 wherein the preferred control range for reaction temperature being below 50° C. and above 10° C., and more preferred temperature conditions being below 40° C. and above 20° C.

20. A process as claimed in claim 1(b), 5 and 19 wherein the most preferred control range for reaction temperature is below 35° C. and above 25° C.

21. A process as claimed in claim 1(b) and 5 wherein the said solvent is hydroxylic solvent.

22. A process as claimed in claim 1(b), 5 and 21 wherein the said solvent is selected from methanol, ethanol, isopropyl alcohol, dimethylformamide, tetrahydrofuran, or water as a single solvent or as mixture of two or more of the said selected solvents.

23. A process as claimed in claim 1(b), 5 and 21 to 22 wherein, a first solvent is selected from dimethyl formamide or tetrahydrofuran in combination with a second solvent selected from methanol,ethanol or isopropyl alcohol in combination with water.

24. A process for purification of 5-[4-[2-[N-methyl-N-(2-pyridyl)amino]ethoxy]phenyl methyl]thiazolidine-2,4-dione (V) as claimed in claim 1(c) wherein the said purification is carried with alcohol under basic complexing conditions.

25. A process as claimed in claims 1(c) and 24 wherein the said preferred alcohol or branched or unbranched aliphatic alcohols.

26. A process as claimed in claims 1(c) and 24 to 25 wherein the most preferred alcohol is lower carbon chain aliphatic alcohol.

27. A process as claimed in claims 1(c) and 24 to 26 wherein the said purification is carried out with the said alcohols such as ethanol, methanol, isopropyl alcohol or t-butanol with basic complexing conditions.

28. A process as claimed in claims 1(c) and 24 to 27 wherein the said purification is carried with the mixture of alcohols alongwith the basic conditions.

29. A process as claimed in claims 1(c) and 24 to 28 wherein a salt of compound (V) is formed under basic complexing conditions.

30. A process as claimed in claims 1(c) and 24 to 29, wherein the said salt formation is carried out using non-aqueous gaseous ammonia as a complexing agent.

31. A process as claimed in claims 1(c) and 24-30, wherein the said complexing agent is purged into alcohol.

32. A process as claimed in claims 1(c) and 24-31, wherein the pH of the solvent with basic conditions is controlled.

33. A process as claimed in claims 1(c) and 24-32, wherein the said pH is not below 8 and not above 12 to achieve optimum basic conditions.

34. A process as claimed in claims 1(c) and 24-33, wherein the most preferred pH is not below 9 and not above 10.

35. A process as claimed in claims 1(c) and 24-34, wherein the said complexing agent is optionally non aqueous liquefied ammonia.

36. A process as claimed in claims 1(c) and 24 to 35, wherein the reaction mixture is neutralized with acid before formation of maleate.

37. A process as claimed in claims 1(c) and 24 to 36 wherein, the said acid is a weak acid.

38. A process as claimed in claims 1(c) and 24 to 37 wherein, the said acid is an organic or inorganic acid.

39. A process as claimed in claims 1(c) and 24 to 38 wherein, the said acid is in diluted form.

40. A process as claimed in claims 1(c) and 24 to 39 wherein, the most preferred said acid is acetic acid for the neutralisation.

41. A process for preparation of 5-[4-[2-N-methyl-N-(2-pyridyl)amino]ethoxy]phenylmethyl]thiozolidine-2,4-dione maleate (VI) as claimed in claim 1(d) wherein the sid process is carried out by treating compound (V) in maleic acid in a mixture of acetone, isopropyl alcohol under controlled temperature.

42. A process as claimed in claim 1(d) and 41 wherein, the ratio of acetone and isopropyl alcohol varies from 5:95 to 95:5.

43. A process as claimed in claims 1(d) and 41 to 42 wherein, the temperature is controlled between 20-40° C.

44. A process as claimed in claims 1(d) and 41 to 43 wherein, the most preferred temperature for the formation of maleic salt is 25-30° C.

45. A process as claimed in 1(c) and claim 5 wherein, the cobalt ion is in the form of cobaltous chloride, the ligand being dimethyl glyoxime and the reducing agent is sodium borohydride.

46. A process as claimed in claim 1, to prepare a compound of the formula V by reacting a compound of the formula IV with a cobalt ion, a ligand and a reducing agent in a suitable solvent wherein a cobalt ion is in the form of Cobaltous Chloride or cobalt diacetate, a ligand is Dimethyl glyoxime and reducing agent is sodium borohydride and wherein, the solvent is a mixture of dimethylformamide, tetrahydrofuran, water and alkalinity imparted by sodium hydroxide.

47. A process as claimed in claim 1, to prepare a compound of the formula V by reacting a compound of the formula IV with a cobalt ion, a ligand and a reducing agent in a suitable solvent wherein a cobalt ion is in the form of cobaltous chloride, a ligand is dimethyl glyoxime and reducing agent is sodium borohydride and wherein, the solvent is a mixture of isopropyl alocohol, dimethylformamide, tetrahydrofuran, water.

48. A process as claimed in claim 1(b) and 5 wherein, the proportion of dimethylformamide: tetrahydrofuran: water in solvent mixture is in the range of 2-3: 3-4:60-70, rest being aqueous alkali.

49. Novel process for the preparation of 5-[4-[2-N-methyl-N-(2-pyridyl)amino]ethoxy]phenyl methyl]thiozolidine-2,4-dione maleate (rosiglitazone maleate) comprises of purification of compound (IV), reduction of compound (IV), isolation of compound (V), and preparation of compound (VI) maleate salt substantially as described in the document individually and collectively with reference to the foregoing examples.
